SEQUENCE LISTING (26 sequences)

SEQ ID NO: 1 - DNA, 13 bp, Artificial Sequence
  LRH-1 response element of the human apo A1 gene promoter.
ctgatccttgaac

SEQ ID NO: 2 - DNA, 13 bp, Artificial Sequence
  Mutated LRH-1 response element of the human apo A1 gene promoter.
ctgattgttgaac

SEQ ID NO: 3 - DNA, 65 bp, Artificial Sequence
  Region B of the human apo A1 gene promoter.
gcagcccccgcagcttgctgtttgcccactctatttgcccagccccagggacagagctgatcctt

SEQ ID NO: 4 - DNA, 87 bp, Artificial Sequence
  Region C of the human apo A1 gene promoter.
gaactcttaagttccacattgccaggaccagtgagcagcaacagggccggggctgggcttatcagcctcccagcccagaccctggct

SEQ ID NO: 5 - DNA, 349 bp, Artificial Sequence
  Apo AI promotor - j04066 (Apo AI gene) 1819- 2167.
gggagacctgcaagcctgcagcactcccctcccgcccccactgaacccttgacccctgccctgcagcccccgcagcttgctgtttgcccactctatttgcccagccccagggacagagctgatccttgaactcttaagttccacattgccaggaccagtgagcagcaacagggccggggctgggcttatcagcctcccagcccagaccctggctgcagacataaataggccctgcaagagctggctgcttagagactgcgagaaggaggtgcgtcctgctgcctgccccggtcactctggctccccagctcaaggttcaggccttgccccaggccgggcctctgggtac

SEQ ID NO: 6 - DNA, 166 bp, Artificial Sequence
  Tk promotor - M80483 (pBLCAT5) 38-204; J02224 (Herpes simplex) 302-462.
tgccccgcccagcgtcttgtcattggcgaattcgaacacgcagatgcagtcggggcggcgcggtccaggtccacttcgcatattaaggtgacgcgtgtggcctcgaacaccgagcgaccctgcagcgacccgcttaacagcgtcaacacgtgccgcagatcacgag

SEQ ID NO: 7 - DNA, 25 bp, Artificial Sequence
  Sense sequence of hCyp7a wt.
gatctcttagttcaaggccagttag

SEQ ID NO: 8 - DNA, 25 bp, Artificial Sequence
  Antisense Sequence of hCyp7a wt.
gatcctaactggccttgaactaaga

SEQ ID NO: 9 - DNA, 25 bp, Artificial Sequence
  Sense Sequence of hCyp 7 a mut.
gatctcttagttcaattccagttag

SEQ ID NO: 10 - DNA, 25 bp, Artificial Sequence
  Antisense Sequence of hCyp 7a mut.
gatcctaactggaattgaactaaga

SEQ ID NO: 11 - DNA, 27 bp, Artificial Sequence
  Sense sequence of LHRE_ApoA1_h_5.
gatccgcagcccccgcagcttgctgta

SEQ ID NO: 12 - DNA, 27 bp, Artificial Sequence
  Antisense sequence of LHRE_ApoAI_h_5.
gatctacagcaagctgcgggggctgcg

SEQ ID NO: 13 - DNA, 32 bp, Artificial Sequence
  Sense sequence of LHRE_ApoA1_h_6.
gatccttgcccactctatttgcccagccccaa

SEQ ID NO: 14 - DNA, 32 bp, Artificial Sequence
  Antisense sequence of LHRE_ApoAI_h_6.
gatcttggggctgggcaaatagagtgggcaag

SEQ ID NO: 15 - DNA, 29 bp, Artificial Sequence
  Sense sequence of LHRE_ApoAI_h_7.
gatccgggacagagctgatccttgaacta

SEQ ID NO: 16 - DNA, 29 bp, Artificial Sequence
  Antisense sequence of LHRE_ApoAI_h_7.
gatctagttcaaggatcagctctgtcccg

SEQ ID NO: 17 - DNA, 29 bp, Artificial Sequence
  Sense sequence of LHRE_ApoAI_h_8.
gatccagcttgctgtttgcccactctata

SEQ ID NO: 18 - DNA, 29 bp, Artificial Sequence
  Antisense sequence of LHRE_ApoAI_h_8.
gatctatagagtgggcaaacagcaagctg

SEQ ID NO: 19 - DNA, 40 bp, Artificial Sequence
  Sense sequence used for mutagenesis of ABCmutLuc+.
ggacagagctgattgttgaactcttaagttccacattgcc

SEQ ID NO: 20 - DNA, 38 bp, Artificial Sequence
  Antisense sequence used for mutagenesis of ABCmutLuc+.
cttaagagttcaacaatcagctctgtccctggggctgg

SEQ ID NO: 21 - DNA, 27 bp, Artificial Sequence
  Sense sequence of FXRRE_ApoA1_h_1.
cagagctgatccttgaactcttaagtt

SEQ ID NO: 22 - DNA, 27 bp, Artificial Sequence
  Antisense sequence of FXRRE_ApoA1_h_1.
aacttaagagttcaaggatcagctctg

SEQ ID NO: 23 - DNA, 27 bp, Artificial sequence
  Sense sequence of FXRRE_ApoA1_h_1_mut.
cagagctgatccttgaagtgttaagtt

SEQ ID NO: 24 - DNA, 27 bp, Artificial sequence
  Antisense sequence of FXRRE_ApoA1_h_1_mut.
aacttaacacttcaaggatcagctctg

SEQ ID NO: 25 - DNA, 29 bp, Artificial sequence
  Sense sequence of LRHRE-ApoA1 mut.
gatccgggacagagctgattgttgaacta

SEQ ID NO: 26 - DNA, 29 bp, Artificial sequence
  Antisense sequence of LRHRE-ApoA1 mut.
gatctagttcaacaatcagctctgtcccg

(19) United States
(12) Patent Application Publication
(10) Pub. No.: US 2008/0207540 A1
(43) Pub. Date: Aug. 28, 2008

(54) Method For the Identification of Compounds Modulating Reverse Transport of Cholesterol

(75) Inventor: Bart Staels, Petit Enghien (BE)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(73) Assignee: Genfit, Loos (FR)

(21) Appl. No.: 10/584,304

(22) PCT Filed: Dec. 23, 2004
(86) PCT No.: PCT/FR04/03373

§ 371 Date: Jun. 23, 2006

(30) Foreign Application Priority Data
Dec. 23, 2003 (FR) 0315273

(51) Int. Cl. A61K 31/70; C12Q 1/68; C07H 21/04; C12N 15/00

(52) U.S. Cl. 514/44; 435/6; 536/23.1; 435/320.1

(57) ABSTRACT

The invention relates to methods and compounds which can modulate reverse cholesterol transport in a mammal and to screening methods enabling the selection, identification and/or characterization of compounds which can modulate reverse cholesterol transport. The invention also relates to cells, vectors and genetic constructs which can be used to implement said methods, in addition to pharmaceutical compositions intended for the treatment of atherosclerosis.

The inventive methods are based on the use of LRH-1 response elements derived from the apo AI gene promoter.

The invention relates to methods and compounds which can modulate reverse cholesterol transport in a mammal and to screening methods enabling the selection, identification and/or characterization of compounds which can modulate reverse cholesterol transport. The invention also relates to cells, vectors and genetic constructs which can be used to implement said methods, in addition to pharmaceutical compositions intended for the treatment of atherosclerosis.

Atherosclerosis is a leading cause of morbidity, mortality, myocardial infarction, cerebral ischemia, cardiovascular disease and peripheral vascularization. Hypercholesterolemia and cholesterol overload in macrophages, which are involved in vascular inflammation, are major contributing factors in atherosclerosis. Currently, hypercholesterolemia is treated through a combination of diet and drugs, for example statins or bile acid sequestering agents. However, novel therapeutic strategies need to be developed to overcome the limitations of existing therapies.

Reverse cholesterol transport, carried out by HDL (High Density Lipoproteins), extracts the cholesterol accumulated in peripheral tissues and ensures the elimination thereof via the liver. In this way it helps protect the body against atherosclerosis. Apolipoprotein AI (apo AI) is a fundamental component of HDL, responsible for the efficacy thereof. In this respect, an increase in the expression of apo AI has a protective effect against atherosclerosis. Apo AI expression is regulated by hormones or by therapeutic agents such as fibrates. It has been shown that nuclear receptors such as HNF4, PPARα or RORα play a critical role in controlling transcription of the apo AI gene. In particular, PPARα is responsible for the increased expression of apo AI induced in humans by fibrates which are used in the clinic for the treatment of dyslipidemias. The identification of new intracellular signal transduction pathways involved in the control of apo AI expression would therefore make it possible to define novel therapeutic strategies which can increase the efficiency of reverse cholesterol transport and thereby protect against atherosclerosis.

The nuclear hormone receptors form a large family of transcription factors the activity of which is modulated by natural and/or artificial ligands. In general, said factors control the expression of their target genes by binding to specific cis-acting response elements and by recruiting accessory proteins necessary for activation of the transcriptional machinery.

The nuclear receptor LRH-1 (Liver Receptor Homolog-1), also known as NR5A2, CPF, hB1F, PHR or FTF, is an orphan receptor for which no ligand has been identified [1]. LRH-1 is a homologue of the drosophila FTZ-F1 receptor whose paralogue in humans is the SF-1 receptor. At least two isoforms, probably arising from alternative use of certain polyadenylation sites, have been identified [2]. LRH-1 expression is confined to the liver, exocrine pancreas and intestine as well as the ovaries [3] and preadipocytes. LRH-1 is expressed early in embryogenesis [4, 5]. LRH-1 does not heterodimerize with RXR but binds as a monomer to a DNA response element with the sequence YCAGGGYCR in which Y=T or C, R=G or A. Several target genes have been identified in the control of the synthesis or transport

of bile acids, the metabolism of steroids [3] and lipoproteins [7, 8] and in the control of transcription or development. LRH-1 also appears to be involved in development of the endoderm [1].

The invention is based on the observation of the role of LRH-1 in the expression of the human gene coding for apolipoprotein AI (apo AI) and on the direct interaction that occurs between LRH-1 and a fragment of the promoter of said gene. The invention is also based on the original observation that overexpression of LRH-1 leads to a stimulation of the activity of the human apo A1 promoter. The invention is also based on the identification of a functional LRH-1 response element at the junction of regions B and C of the human apo A1 gene promoter (as defined in [16]) and on the characterization of the sequence thereof.

The invention thus demonstrates for the first time a modulation of apo A1 production by the LRH-1 nuclear receptor. The invention thereby provides novel targets and novel approaches for the search for compounds which can regulate the expression of said protein, the activity of HDL or reverse cholesterol transport. The invention also provides methods for increasing reverse cholesterol transport based on the use of a compound which modulates the binding of LRH-1 to the apo A1 promoter and/or the effect thereof on transcription of the human apo A1 gene

The invention also provides screening methods to enable the selection, identification or characterization of therapeutic substances which can modulate the expression of the human apo A1 gene and/or the activity of HDL and/or reverse cholesterol transport.

According to a particular embodiment, the screening methods of the invention more particularly include the following steps:
contacting one or more compounds with a nucleic acid construct comprising at least one LRH-1 response element of the human apo A1 gene promoter or a functional variant thereof,
determining the possible binding of said compounds to the response element(s), and
optionally comparing the preceding measurement with a measurement carried out in the same conditions but with a nucleic acid construct comprising at least one mutated copy of an LRH-1 response element of the human apo A1 gene promoter.

Generally, said contact is carried out in conditions that allow said compounds to bind to said response element.

In a preferred manner, the inventive method comprises:
contacting a test compound with a nucleic acid construct comprising, as the only LRH-1 response element, at least one copy of the LRH-1 response element of the human apolipoprotein AI gene promoter containing the following sequence (SEQ ID NO: 1) 5′-CTGATCCTTGAAC-3′, and
determining the possible binding of said test compound to the response element, and
optionally comparing the preceding measurement with a measurement carried out in the same conditions but with a nucleic acid construct comprising at least one mutated copy of an LRH-1 response element of the human apo A1 gene promoter.

According to a particular embodiment of the inventive method, the conditions which allow said compounds to bind said LRH-1 response element(s) comprise the presence of the LRH-1 receptor, generally exogenous, (for example in monomeric form) or a functional equivalent, and the determination of the possible binding of said test compound to the LRH-1 response element and/or to the complex formed by the binding of LRH-1 to its response element.

According to another particular embodiment (test of transcriptional activity), one measures the effect of one or more test compounds, optionally in the presence of the exogenous LRH-1 receptor or a functional equivalent thereof, on the transcriptional activity of a promoter comprising at least one LRH-1 response element according to the invention. Said test is preferably carried out in a cellular system, by determining the expression of a reporter gene placed under the control of said promoter, particularly in a cell comprising exogenous LRH-1 or an equivalent thereof and/or comprising a ligand of LRH-1 or a functional equivalent thereof. According to another embodiment, the test is carried out in a cell comprising (e.g., expressing, in a natural or recombinant manner) the LRH-1 receptor or a functional equivalent thereof.

A preferred embodiment of the invention is the use, optionally in the presence of exogenous LRH-1 or an equivalent thereof, of an expression cassette combining one or more LRH-1 response elements, according to the invention, with a reporter gene.

Avantageously, said reporter gene is placed under the control of a promoter comprising at least one copy of said response elements, for example, the apo A1 promoter or variants or fragments of same. Any gene known to those skilled in the art whose activity or presence in biological extracts can be easily measured can be used as reporter gene in order to carry out the screening method.

According to a particular embodiment of the invention, the screening method comprises the steps of:
contacting a test compound with a host cell containing a reporter gene expression cassette, said cassette comprising a reporter gene placed under the control of a promoter comprising, as the only LRH-1 response element, at least one copy of the LRH-1 response element of the human apolipoprotein AI gene promoter containing the following sequence (SEQ ID NO: 1): 5′-CTGATCCTTGAAC-3′, and
determining the effect of the presence of the test compound on the binding of LRH-1 to the response element or on the expression of the reporter gene.

Preferably, the host cell contains an exogenous LRH-1 receptor or a functional equivalent thereof and/or a ligand of LRH-1 or a functional equivalent thereof.

According to another particular embodiment, the method comprises determining the level of expression of the reporter gene in the presence of the test compound and in the absence of said compound, an increase or a decrease in the expression level indicating the ability of the test compound to modulate reverse cholesterol transport.

The compounds that can be identified by the method accodring to the invention can be compounds of different nature, structure and origin, in particular biological compounds, nuclear factors, cofactors, chemical, synthetic compounds, and the like, which are capable of modifying the activity of LRH-1. They can also be libraries, particularly combinatorial libraries, chemical libraries or libraries of proteins, peptides or nucleic acids, for example clones coding for one or more DNA-binding proteins, peptides or polypeptides.

The methods according to the invention can be used for the selection, identification or characterization of compounds which can modify the binding of LRH-1 and/or its cofactors to one and/or the other of its response elements and/or which can modify (i.e., increase or decrease) the expression of the gene coding for human apo A1 and hence the expression of human apo AI and/or which can modulate the activity of HDL and/or which can modulate reverse cholesterol transport.

The invention further describes the use of the compounds selected in this manner, for preparing a composition for modulating reverse cholesterol transport or the activity of HDL, and the corresponding treatment methods.

For easier comprehension of the application, the following definitions are provided, which clarify or complete their usual meaning.

“Apolipoprotein AI” or apo AI : Apolipoprotein Al is a protein containing 243 amino acids with a globular amino-terminal end and a carboxy terminal end which can bind lipids [9]. Said protein is a major constituent of high density lipoproteins and plays a fudamental role in reverse cholesterol transport [10, 11]. The gene, the cDNA and the MRNA of apo AI have been cloned and sequenced [12-14], and can be found in the Genbank® data base (for example on the Internet at: http ://www.ncbi.nlm.nih.gov) under the access numbers: NM_{—}000039, M20656 (promoter) and J00098.

“High Density Lipoprotein (HDL)”: HDL particles are high density lipoproteins (1.063-1.21 g/ml) reputed to have a protective role against atherosclerosis mainly due to their ability to extract cholesterol from peripheral cells and promote the return thereof to liver where it is eliminated [10]. Apo AI is the major protein constituent of HDL, accounting for up to 70% of the proteins. HDL also comprise apo AII, apo CI, apo CII, apo CIII and apo E in smaller proportions.

“LRH-1”: The LRH-1 receptor has been isolated, characterized and sequenced in humans and rats. The MRNA sequence is also available in the Genbank® data base under the access numbers NM_{—}003822, NM_{—}030676 and NM_{—}021742 for humans, mice and rats, respectively. The region of LRH-1 involved in DNA binding (“DNA binding domain”) is principally comprised between residues Glu38-Asp113 of the human protein (corresponding to 319-546 in NM_{—}003822) or between residues Glu105-Asp180 of the mouse protein.

The term “functional equivalent” which refers to the LRH-1 receptor denotes any polypeptide derived from the structure of the LRH-1 receptor and conserving the ability to bind the response element particularly any response element having the sequence SEQ ID NO: 1 or functional variants of same. The functional equivalents can be natural variants (polymorphism, splicing, etc.), fragments, mutants, deletants, and the like. Preferably, they are polypeptides comprising at least one amino acid region displaying at least 60% identity to the LRH-1 receptor, preferably at least 75% and even more preferably at least 90-95%. The term also includes fragments of the LRH-1 receptor, in particular fragments containing the DNA binding site of the LRH-1 receptor.

The term “reverse transport” is used to designate the physiological mechanism, sometimes defective, through which excess cholesterol in peripheral tissues is processed by high density lipoproteins, or HDL (High Density Lipoprotein), then transported to the liver for elimination.

### A. Identification of an LRH-1 Response Element.

The invention demonstrates the involvement and the mechanism of action of LRH-1 in the regulation of apo AI expression and, so doing, in the regulation of reverse cholesterol transport. Overexpression of LRH-1 results in an increase in the activity of the apo AI promoter. The invention also reveals the exact sequence of the LRH-1 response element, in the promoter of the gene coding for human apo AI.

The invention also relates to particular constructs, in particular of nucleic acids comprising LRH-1 response elements, and to cassettes, vectors and recombinant cells containing same.

Thus the invention provides the sequence (SEQ ID NO: 1) of the LRH-1 response element, initially identified in the human apo AI gene promoter, responsible for an interaction between LRH-1 and the apo AI promoter and for the regulation by LRH-1 of apo AI expression.

Three different functional regions have been identified in the apo AI promoter [15]. Herein, the functional domains of the apolipoprotein AI gene promoter are named A, B and C according to the previously defined nomenclature [16].

Thus, the presence of regions B and C (SEQ ID NO : 3 and 4) induces an increase by LRH-1 in the expression of a reporter gene (see examples 1, 2, 5 and 6).

A particular object of the invention is a nucleic acid comprising sequence SEQ ID NO: 1 which follows:

5′-CTGATCCTTGAAC-3′, or a functional variant thereof (“LRH-1 response element”).

Another object of the invention is based on a nucleic acid construct comprising an LRH-1 response element such as defined hereinabove. In particular it can be an expression cassette comprising at least one copy of a response element such as defined hereinabove.

The invention also has as object an expression cassette comprising at least one copy of the nucleic acid fragment comprising or preferably characterized by the following sequence SEQ ID NO: 1:

5′-CTGATCCTTGAAC-3′, or a functional variant thereof, and a promoter, selected in the group consisting of the CMV immediate early promoter and the PGK promoter, associated with a reporter gene placed under the control of said promoter.

In particular, said expression cassette can be used for in vitro screening of compounds which can modulate the activity of HDL.

The invention further relates to any artificial or chimeric promoter comprising an LRH-1 response element such as defined hereinabove.

The functional variants of the response element according to the invention can be any derivative or fragment of the native sequence which conserves the ability ot bind the LRH-1 receptor. Generally, the variants conserve at least 50% of the residues of the native sequence described herein. Classically, the variants contain modifications affecting fewer than 5 nucleotides in the sequence in question. Preferably, it is a sequence displaying at least 60% identity, preferably at least 75% and even more preferably at least 90% identity to the native sequence described in this application. The variants can contain different types of modifications such as one or more point mutations or not, additions, deletions and/or substitutions.

Said modifications can be introduced by conventional physical, chemical or molecular biological methods, such as in particular site directed mutagenesis or, more practically, by artificial synthesis of the sequence in a synthesizer.

The variants can be tested for their ability to bind LRH-1 in different ways, and in particular:
(i) by contacting the test sequence with the LRH-1 receptor (for example in an acellular test), and detecting the formation of a complex (for example by gel shift);
(ii) by inserting the test sequence in an expression cassette comprising a minimal promoter and a reporter gene, introducing the cassette in a cell, and detecting (assaying as the case may be) the expression of the reporter gene in the presence and absence of LRH-1;
(iii) by any other technique known to those skilled in the art, enabling the demonstration of an interaction between a nucleic acid and a protein, for example.

The invention also has as object inactive variants of the aforementioned response elements, in particular variants essentially unable to bind the LRH-1 receptor. Such variants are exemplified in particular by sequence SEQ ID NO: 2.

Said inactive variants can be prepared and tested in the conditions described hereinabove for functional variants.

Advantageously, the variants according to the invention can hybridize with the sequence SEQ ID NO: 1 or a part thereof.

### B. Methods for the Selection, Identification and Characterization of Compounds which Modulate Reverse Cholesterol Transport.

The invention describes methods for the identification of compounds which modulate (i.e., increase or decrease) reverse cholesterol transport. Said compounds can act by altering the binding of LRH-1 to its ligand(s) or to its corepressor(s) and coactivator(s), etc. Or else they can modify, or suppress, the binding of LRH-1 alone or of LRH-1 and its cofactors, to its response element(s) and thus modify the expression of the human apo AI gene. Binding of LRH-1 to the response element present at the junction of regions B and C of the apo AI promoter (SEQ ID NO: 3 and 4) thereby increases the transcription of the human apo AI gene and stimulates reverse cholesterol transport. The use of compounds which can increase LRH-1 binding to said response element, where LRH-1 plays the role of activator, therefore makes it possible to increase the transcription of the human apo AI gene and to stimulate reverse cholesterol transport.

The invention thus describes novel methods for the selection, identification or characterization of compounds which can increase reverse cholesterol transport.

### 1. Methods Based on Expression Screening.

The invention relates to a method for the selection, identification or characterization of compounds which can modulate reverse cholesterol transport, which comprises:
(i) contacting a test compound with a host cell containing a reporter gene expression cassette, said cassette comprising a reporter gene placed under the control of a promoter comprising at least one copy of the LRH-1 response element of the human apo AI gene promoter or a functional variant thereof, and
(ii) determining the expression of the reporter gene.

In a preferred manner, the inventive method comprises:
(i) contacting a test compound with a host cell containing a reporter gene expression cassette, said cassette comprising a reporter gene placed under the control of a promoter comprising, as the only LRH-1 response element, at least one copy of the LRH-1 response element of the human apolipoprotein AI gene promoter containing the following sequence (SEQ ID NO: 1): 5′-CTGATCCTTGAAC-3′, and
(ii) determining the effect of the presence of the test compound on the binding of LRH-1 to the response element or on the expression of the reporter gene.

The invention also relates to a method for the selection, identification or characterization of compounds which can modulate reverse cholesterol transport, which comprises:
(i) contacting, in the presence of the exogenous LRH-1 receptor or a functional equivalent thereof, a test compound with a host cell containing a reporter gene expression cassette, said cassette comprising a reporter gene placed under the control of a promoter comprising at least one copy of an LRH-1 response element of the human apo AI gene promoter or a functional variant thereof, and
(ii) determining the effect of the presence of the test compound on the binding of LRH-1 to the response element or on the expression of the reporter gene.

Even more preferably, the inventive method comprises:
(i) contacting, in the presence of the exogenous LRH-1 receptor or a functional equivalent thereof, a test compound with a host cell containing a reporter gene expression cassette, said cassette comprising a reporter gene placed under the control of a promoter comprising, as the only LRH-1 response element, at least one copy of the LRH-1 response element of the human apolipoprotein AI gene promoter containing the following sequence (SEQ ID NO: 1): 5′-CTGATCCTTGAAC-3′, and
(ii) determining the effect of the presence of the test compound on the binding of LRH-1 to the response element or on the expression of the reporter gene.

More specifically, the methods according to the invention provide for contacting a test compound with a nucleic acid construct or an expression cassette comprising at least one copy of an LRH-1 response element (SEQ ID NO: 1).

A particular object of the invention relates to an expression cassette comprising at least one copy of the nucleic acid fragment SEQ ID NO: 1, and a promoter associated with a reporter gene placed under the control of said promoter.

Another particular object of the invention relates to an expression cassette comprising at least one mutated copy of the nucleic acid fragment SEQ ID NO: 1, and a promoter associated with a reporter gene placed under the control of said promoter.

In a particular embodiment of the inventive methods, it is additionally provided to compare the possible effects, determined by one of said methods, with the possible effects determined by a method carried out under the same conditions but with a nucleic acid construct comprising at least one inactive variant (for example, a mutated copy) of an LRH-1 response element of the human apo AI gene promoter (SEQ ID NO : 2) or a functional variant thereof.

According to another particular embodiment of the inventive methods, the host cell contains a ligand of LRH-1 or a functional equivalent thereof. The inventive methods can be carried out with different types of cells, promoters, reporter genes, and in different conditions, as described hereinbelow.
a) Contacting the Compounds with the Host Cell.

Certain screening methods, described by the invention, provide for a step of contacting the test compound, optionally in the presence of the exogenous LRH-1 receptor or a functional equivalent thereof, with host cells, in specific conditions which allow to determine the expression in said cells of a reporter gene and thereby to obtain information on the effect of the test compound.

Preferably, the LRH-1 receptor is introduced or added artificially so as to have at least twice the quantity of endogenous LRH-1. It can be an equivalent of LRH-1, namely, any nucleic acid sequence displaying at least 60% identiy to that of the LRH-1 receptor, preferably at least 75% and even more preferably at least 90-95%.

Classically, the effect of the test compound is compared in terms of the level of expression of the reporter gene determined in the absence of said compound (and/or with a mutated reponse element).

According to a particular embodiment, the inventive methods comprise determining the level of reporter gene expression in the presence of the test compound and in the absence of said compound, an increase or a decrease in reporter gene expression indicating the ability of the test compound to modulate reverse cholesterol transport.

In a preferred embodiment, said cells can be mammalian cells (hepatocytes, fibroblasts, endothelial, muscle cells, etc.). Even more preferably, said cells can be human cells. They can also be primary cultures or established cell lines. In another embodiment, it is also possible to use prokaryotic cells (bacteria), yeast cells (Saccharomyces, Kluyveromyces, etc.), plant cells, and the like.

The compounds can be contacted with the cells at different times, depending on their effect(s), their concentration, the nature of the cells and technical considerations. Contact can be carried out on any suitable support and in particular on a plate, in a tube or a flask. Generally, contact is carried out in a multiwell plate which makes it possible to conduct a large number of different tests simultaneously. Typical supports include microtitration plates and more particularly plates having 96 or 384 wells (or more), which are easy to manipulate and on which visualization can be accomplished by a classical stimulation.

Depending on the support and the nature of the test compound, variable amounts of cells can be used when implementing the aformentioned methods. Classically, 10³ to 10⁶ cells are contacted with a type of test compound, in a suitable culture medium, and preferably between 10⁴ and 10⁵ cells. By way of example, in a 96-well plate, 10⁵ cells can be incubated in each well with a desired quantity of test compound. In a 384-well plate, fewer than 10⁵ cells and typically between ×10⁴ and 4×10⁴ cells are generally incubated in each well with the test compound.

The quantity (or the concentration) of test compound can be adjusted by the user according to the type of compound (its toxicity, ability to penetrate cells, etc.), the number of cells, the incubation time, etc. Generally, the cells are exposed to test compounds ranging in concentration from 1 nM to 1 mM. Of course it is possible to test other concentrations without deviating from the invention. Furthermore, each compound can be tested in parallel at different concentrations.

Different adjuvants and/or vectors and/or products that facilitate penetration of the compounds in the cells such as liposomes, cationic lipids, polymers, penetratin, Tat PDT, peptides arising from adenovirus (penton or fibers) or other viruses, etc. can also be used where necessary.

Contact is maintained for 5 to 72 hours, generally between 12 and 48 hours. In fact, the cells and the various reagents should preferably remain in contact long enough to allow de novo synthesis of the expression product of the reporter gene. In a preferred manner, the incubation time is approximately 36 hours.
b) Determination of the Activity of the Compounds.

The method proposed in the invention for the selection, identification or characterization of compounds which can modulate reverse cholesterol transport provides for the transformation of host cells with a reporter gene expression cassette. In particular, said reporter gene can be any gene coding for and expressing a product whose activity or presence, in biological extracts, can be measured, that is, detected or assayed, or whose transcription product can be measured. Examples include the gene coding for human apo AI itself, or else the gene coding for luciferase and more particularly for firefly or Renilla luciferase, for secreted alkaline phosphatase, galactosidase, lactamase, chloramphenicol acetyltransferase (CAT), human growth hormone (hGH), P-glucuronidase (Gluc), green fluorescent protein (GFP), etc. It is understood that the term “gene” designates, in the broad sense, any nucleic acid, in particular a cDNA, gDNA, synthetic DNA, an RNA, and the like.

The reporter gene, whatever it may be, is placed under the control of a promoter comprising at least one copy of an LRH-1 response element such as defined hereinabove.

The reporter gene can therefore be placed under the control of any promoter the sequence of which comprises the sequence SEQ ID NO: 1 or a functional variant thereof. Said particular sequence can be present in one or more copies in the promoter (preferably 1 to 10 and even more preferably 1 to 6), upstream, downstream or internally, in the same orientation or in the opposite orientation. Preferably, the promoter according to the invention comprises, as the only LRH-1 response element, at least one copy of the LRH-1 response element of the human apolipoprotein AI gene promoter containing the following sequence (SEQ ID NO: 1): 5′-CTGATCCTTGAAC-3′ or a functional variant thereof.

Preferably, it is a promoter whose differential activity in the absence and in the presence of LRH-1 or a functional equivalent thereof, can be detected.

To prepare the inventive promoter, the LRH-1 response element can be associated with a transcriptional minimal promoter. The minimal promoter is a transcriptional promoter having an activity which is low or nonexistent, and which can be increased in the presence of a transcriptional activator (the interaction of LRH-1 with the junction of regions B and C). A minimal promoter can therefore be a promoter which is naturally weak in mammalian cells, that is, producing a nontoxic and/or insufficient expression to obtain a marked biological effect. Avantageously, a minimal promoter is a construct prepared from a native promoter, by deleting region(s) nonessential to transcriptional activity. For instance, it is preferably a promoter comprising essentially a TATA box, generally less than 160 nucleotides in size, centered around the transcription initiation codon. A minimal promoter can thus be prepared from strong or weak viral or cellular promoters, such as for example the promoter of the thymidine kinase (TK) gene of herpes virus (HSV-TK), the CMV immediate early promoter, the PGK promoter, the promoter of the gene coding for human apolipoprotein AI, the SV40 promoter, etc. The minimal promoter can have sufficiently high activity to enable the identification of compounds which increase activation by LRH-1, via regions B and C for example.

The promoter (P), the LRH-1 response element (RE) and the reporter gene (RG) are arranged in a functional manner in the expression cassette, that is, so that the minimal promoter controls the expression of said gene and so that its activity is regulated by LRH-1. Generally, therefore, said regions are arranged in the following order, in the 5′→3′ orientation: RE-P-RG. However, any other functional arrangement can be envisioned by those skilled in the art without deviating from the invention.

In addition, the different aforementioned functional domains can directly flank each other, or be separated by nucleotides which do not significantly affect the functional character of the expression cassette or which confer improved characteristics or performance of the system (amplifier, silencer, intron, splicing site, etc.).

The method for the selection, identification and characterization of compounds which can modulate reverse cholesterol transport provides for a step of determining the expression of the reporter gene. This may be a determination of transcriptional activity. To this end, total RNA is extracted from the cells cultured in the experimental conditions on the one hand, and in a control situation on the other hand. Said RNA is used as probe to analyze changes in the expression of the reporter gene(s), for example.

It may also be a matter of visualizing the expression of the reporter gene with the help of a suitable subtrate. Said visualization can be carried out by different techniques the nature of which depends on the type of reporter gene used. For example, the measurement can correspond to an optical density, a fluorescent or luminescent emission in the case where the gene coding for β-galactosidase or luciferase is used as reporter gene.

In a particular embodiment, the expression of the reporter gene is measured in terms of the level of hydrolysis of the expression product of the reporter gene. For example, many substrates can be used to evaluate the expression of β-lactamase. In particular they can be any product containing a β-lactam nucleus and the hydrolysis of which can be measured. Preferred substrates are those specific of β-lactamase (i.e., they are generally not hydrolyzed in mammalian cells in the absence of β-lactamase), those which are not toxic to mammalian cells and/or whose hydrolysis product can be easily measured, for example by methods based on fluorescence, radioactivity, an enzymatic activity or any other method of detection.

Even more preferred substrates are ratiometric substrates. The hydrolysis of said substrates can be directly correlated with the activity of the expression product of the reporter gene by the number of cells. A specific and nontoxic ratiometric substrate that can be used in the invention is CCF2-AM.

The concentration of the substrate can be adjusted by those skilled in the art according to the number of cells, for example. The cells are generally kept in contact with the substrate for approximately 60 minutes.

The presence of the reporter gene product (or the hydrolysis product of the substrate) can be determined by conventional methods known to those skilled in the art (fluorescence, OD, luminescence, FRET (see WO 0037077), SPA, biochips, immunological methods, etc.).

Generally, one determines the activity of a test compound in a cell and this effect is compared with the level of activity in the absence of test compound or with a mean value determined in the absence of any test compound.

The measurement of hydrolysis primarily involves measuring (or determining the relative quantity) of hydrolysis product contained in each reaction sample. Said measurement can be carried out by different methods known to those skilled in the art, including detection of fluorescence, radioactivity, a color, an enzymatic activity, an antigen-antibody immune complex, etc. In a preferred manner, the hydrolysis product is detected and quantified by a fluorescence detection method. Different fluorochromes can thus be used and measured in the cell samples.

A secondary test whereby the selection of the compounds can be validated in animals can also be carried out by determining the quantity of HDL expressed or by determining a significant variation in reverse cholesterol transport in cells treated with said compounds in comparison with untreated cells. It is also possible to measure plasma cholesterol and/or determine hepatic expression of apo AI.

According to a preferred embodiment of the invention, the host cell also comprises an LRH-1 ligand. The term “LRH-1 ligand” also applies to transcription factors, coactivators and corepressors, as well as to other polypeptides involved in the regulatory machinery of gene expression. For example, it may be other receptors like RXR or nuclear hormone receptors.

According to another preferred embodiment of the invention, and as indicated earlier, a host cell comprising the LRH-1 receptor or a functional equivalent is used in the inventive methods. The presence of the LRH-1 receptor mimics a physiological situation and enables the identification, through the aforementioned methods, of compounds which can modulate interactions between LRH-1 and one and/or the other of its response element(s), such as disclosed in the invention, or between LRH-1 and one or more ligand(s) of LRH-1.

The inventive methods enable the determination of the level of expression of the reporter gene, according to one of the methods known to those skilled in the art described hereinabove, in the presence of the test compound and/or in the absence of said compound, an increase or a decrease in the level of reporter gene expression indicating the ability of the test compound to modulate reverse cholesterol transport.

The invention can therefore be carried out with the help of a construct, cassette or cell according to the invention used for in vitro screening of compounds which can modulate the activity of HDL.

As indicated earlier, said methods enable the rapid and simultaneous screening of many test compounds on one or more cell populations (mammalian cells, human cells such as for example hepatocytes, prokaryotic cells, etc.). Said methods are predictive, can be automated and are adapted to the selection, identification and characterization of said compounds.

A particular embodiment of the screening method makes use of classical methods for identifying clones which express DNA binding proteins. For example it may be a matter of screening cDNA expression libraries in λgt11 or using the so-called “One Hybrid” or “Phage Display” method, or else carrying out a purification by affinity chromatography. The isolated protein(s) are then sequenced.

### 2. Methods Based on a Binding Test.

The invention also relates to a method for the selection, identification or characterization of compounds which can modulate (i.e., increase or decrease) reverse cholesterol transport, based on measuring the binding of a test compound to one or more response elements. Said method more particularly comprises:
contacting a test compound with a nucleic acid construct comprising at least one copy of an LRH-1 response element of the human apo AI gene promoter or a functional variant thereof, and
determining the possible binding of said test compound to the response element.

Preferably, the inventive method comprises:
contacting a test compound with a nucleic acid construct comprising, as the only LRH-1 response element, at least one copy of the LRH-1 response element of the human apolipoprotein AI gene promoter containing the following sequence (SEQ ID NO: 1): 5′-CTGATCCTTGAAC-3′, and
determining the possible binding of said test compound to the response element.

Another inventive method comprises
contacting, in the presence of the exogenous LRH-1 receptor or a functional equivalent thereof, a test compound with a nucleic acid construct comprising at least one copy of an LRH-1 response element of the human apo AI gene promoter or a functional variant thereof, and
determining the binding of the test compound to the LRH-1 response element(s) and/or to the complex formed by the binding of LRH-1 to its response element(s).

Even more preferably, the inventive method comprises:
contacting, in the presence of the exogenous LRH-1 receptor or a functional equivalent thereof, a test compound with a nucleic acid construct comprising, as the only LRH-1 response element, at least one copy of the LRH-1 response element of the human apolipoprotein AI gene promoter containing the following sequence (SEQ ID NO: 1): 5′-CTGATCCTTGAAC-3′, and
determining the possible binding of said test compound to the LRH-1 response element and/or to the complex formed by the binding of LRH-1 to its response element.

A preferred embodiment of the invention consists in establishing the ability of said test compound to modulate the binding of LRH-1 to the response element, by determining the quantity of LRH-1 bound in the presence of the test compound compared with this quantity in the absence of the test compound. For instance, a competition test using the FP (Fluorescence Polarization) method, known to those skilled in the art, can be efficiently used for said determination.

A test compound which can modulate the binding of LRH-1 to the response element can subsequently be tested for its ability to modulate the expression of a reporter gene and/or reverse cholesterol transport, according to one of the methods described hereinabove.

The binding of the test compound to at least one LRH-1 response element can be demonstrated by gel shift, by electrophoresis of the heterodimers formed following the implementation of the aforementioned method. In fact, some test compounds can contain a DNA binding site largely identical to that of LRH-1 and thereby compete with the latter.

Electrophoresis allows to directly distinguish heterodimers composed of LRH-1/LRH-1 response element, from heterodimers composed of test compound/LRH-1 response element and LRH-1 response elements.

Other luminescence-based methods or using the FRET (Fluorescence Resonance Energy Transfer) technique, well known to those skilled in the art, or the SPA (Scintillation Proximity Assay) technique, can be used in the invention to determine the possible binding of the test compound to one and/or the other of the LRH-1 response elements.

In a particular embodiment, the nucleic acid construct comprises at least 1 copy, preferably from 2 to 5 copies of the sequence SEQ ID NO:1 or a functional variant thereof. The test compounds which can activate (that is, at least partially increase) LRH-1 binding to said construct enable the activation of reporter gene expression and represent candidates for stimulation of reverse cholesterol transport.

According to a particular embodiment of the invention, it is also provided to compare the possible effects, determined by one of said methods, with the possible effects, determined by a method carried out in the same conditions but with a nucleic acid construct comprising at least one inactive variant (for example, a mutated copy) of an LRH-1 response element of the human apo AI gene promoter (SEQ ID NO: 2) or a functional variant of same. In a preferred manner, the nucleic acid construct contains at least one mutated copy of the LRH-1 response element of the promoter of the human apolipoprotein AI gene, containing the following sequence (SEQ ID NO:1): 5′-CTGATCCTTGAAC-3′, said mutated copy essentially being unable to bind the LRH-1 receptor.

### C. Activity of HDL and Apolipoprotein AI.

The methods described hereinabove for the selection, identification or characterization of compounds which can modulate (i.e., increase or decrease) the expression of a reporter gene and/or reverse cholesterol transport are preferably used for screening compounds which can increase reverse cholesterol transport and, according to another embodiment of the invention, can be used for the selection, identification or characterization of compounds which can modulate the activity of HDL and/or the expression of po AI.

### D. Test Compounds.

The invention can be used with any type of test compound. For instance, the test compound can be any product which is isolated or in a mixture with other products. The compound can be defined in terms of its structure and/or composition or not be defined. For example, the compound can be an isolated and structurally defined product, an isolated product with undefined structure, a mixture of known and characterized products or an undefined composition comprising one or more products. One or more compounds can thus be tested, in a mixture or separately. For example, said undefined compositions can be samples of tissues, biological fluids, cell supernatants, plant preparations, and the like. The test compounds can be inorganic or organic products and in particular a polypeptide (or a protein or a peptide), a nucleic acid, a lipid, a polysaccharide, a chemical compound or a biological compound such as a nuclear factor, a cofactor or any mixture or derivative of same. The compound can be natural or synthetic in origin and can include a combinatorial library, a clone or a library of nucleic acid clones expressing one or more DNA-binding peptides, etc.

The invention is particularly suited to the selection, identification or characterization of a large number of compounds. This simple and efficient screening can be accomplished in a very short period of time. In particular, the aforementioned methods can be partly automated, thereby enabling an efficient and simultaneous screening of many different compounds, either in the form of a mixture or separately.

### E. Use of the Identified Compounds.

The compounds identified according to the invention have advantageous properties for a therapeutic use, in particular in the field of atherosclerosis. The invention thus provides for the use of a compound which can modulate (i.e., increase or decrease) the binding of LRH-1 and/or its cofactors to response elements in the promoter of the gene coding for human apo AI or a functional variant thereof (in particular to the sequence SEQ ID NO:1 or a functional variant thereof), for preparing a composition intended to modulate (i.e., increase or decrease) reverse cholesterol transport. According to another embodiment of the invention, said use can be intended to modulate (I;e., increase or decrease) the activity of HDL or to modulate the expression of apo AI.

Another embodiment of the invention provides for the use of a compound which can modulate (i.e., increase or decrease) the effect of LRH-1 on transcription of the human apo AI gene or a functional variant thereof, for preparing a composition intended to modulate (i.e., increase) reverse cholesterol transport and/or to modulate (i.e., increase or decrease) the activity of HDL.

In a preferred embodiment of the invention it is a chemical compound or a biological compound. In another preferred embodiment, it is a nuclear factor or a cofactor. In an even more preferred embodiment, it is a clone expressing one or more DNA-binding polypeptide(s). In a general manner, it can be any compound which is selected, identified or characterized according to one of the aforementioned methods.

The invention encompasses the use of any compound (or derivatives of said compounds) which is selected, identified or characterized according to one of the aforementioned methods, in the scope of the invention, as target of experimental research or for the production of pharmaceutical compositions intended to increase reverse cholesterol transport or to treat hypercholesterolemia, atherosclerosis, lipid disorders and/or cardiovascular diseases, as well as said pharmaceutical compositions.

Other advantages and applications of the invention will become apparent in the following examples, which are given for purposes of illustration and not by way of limitation.

### LEGENDS OF FIGURES

FIG. 1: Effect of LRH-1 overexpression on the activity of the human apo AI promoter in HepG2 cells (RLU: relative luminescence unit).

FIG. 2: Effect of LRH-1 overexpression on the activity of the human apo AI promoter in RK13 cells (RLU: relative luminescence unit).

FIG. 3: Gel shift showing the identification of an LRH-1 response element located at the junction of regions B and C of the human apo AI promoter. The separated complexes, appearing on the electrophoresis gel, are identified in example 3.

FIG. 4 A/B: Gel shift showing the identification of an LRH-1 response element comprised in the fragment −144/−122 of the human apo AI promoter.

FIG. 5: Effect of LRH-1 overexpression on the activity of the human apo AI promoter, mutated or not, in HuH7 cells (RLU: relative luminescence unit).

FIG. 6: Effect of LRH-1 overexpression on the activity of different mutants of the human apo AI promoter in HuH7 cells.

FIG. 7 A/B: The LRH-1 binding site in the human apo AI gene promoter is different from the FXR binding site in the human apo AI gene promoter.

### SEQUENCES

SEQ ID NO:1
(LRH-1 response element of the human apo AI gene
promoter):
5′-CTGATCCTTGAAC-3′
SEQ ID NO:2
(Mutated LRH-1 response element of the human apo
AI gene promoter):
5′-CTGATTGTTGAAC-3′
SEQ ID NO:3
(Region B of the human apo AI gene promoter):
5′-GCAGCCCCCGCAGCTTGCTGTTTGCCCACTCTATTTGCCCAGCCCCA
GGGACAGAGCTGATCCTT-3′
SEQ ID NO:4
(Region C of the human apo AI gene promoter):
5′-GAACTCTTAAGTTCCACATTGCCAGGACCAGTGAGCAGCAACAGGGC
CGGGGCTGGGCTTATCAGCCTCCCAGCCCAGACCCTGGCT-3′
SEQ ID NO:5
(Apo AI promoter j04066 (apo AI gene) 1819-2167):
5′-gggagacctgcaagcctgcagcactcccctcccgcccccactgaacc
cttgacccctgccctgcagcccccgcagcttgctgtttgcccactctatt
tgcccagccccagggacagagctgatccttgaactcttaagttccacatt
gccaggaccagtgagcagcaacagggccggggctgggcttatcagcctcc
cagcccagaccctggctgcagacataaataggccctgcaagagctggctg
cttagagactgcgagaaggaggtgcgtcctgctgcctgccccggtcactc
tggctccccagctcaaggttcaggccttgccccaggccgggcctctgggt
ac-3′
SEQ ID NO:6
(TK promoter M80483 (pBLCAT5) 38-204; J02224
(Herpes simplex) 302-462):
5′-tgccccgcccagcgtcttgtcattggcgaattcgaacacgcagatgc
agtcggggcggcgcggtccaggtccacttcgcatattaaggtgacgcgtg
tggcctcgaacaccgagcgaccctgcagcgacccgcttaacagcgtcaac
acgtgccgcagatcacgag-3′
SEQ ID NO:7
(sense sequence of hCyp7a wt):
5′-GATCTCTTAGTTCAAGGCCAGTTAG-3′
SEQ ID NO:8
(antisense sequence of hCyp7a wt):
5′-GATCCTAACTGGCCTTGAACTAAGA-3′
SEQ ID NO:9
(sense sequence of hCyp7a mut):
5′-GATCTCTTAGTTCAATTCCAGTTAG-3′
SEQ ID NO:10
(antisense sequence of hCyp7a mut):
5′-GATCCTAACTGGAATTGAACTAAGA-3′
SEQ ID NO:11
(sense sequence of LHRE-ApoA1_h_5):
5′-GATCCGCAGCCCCCGCAGCTTGCTGTA-3′
SEQ ID NO:12
(antisense sequence of LHRE_ApoA1_h_5):
5′-GATCTACAGCAAGCTGCGGGGGCTGCG-3′
SEQ ID NO:13
(sense sequence of LHRE_ApoA1_h_6):
5′-GATCCTTGCCCACTCTATTTGCCCAGCCCCAA-3′
SEQ ID NO:14
(antisense sequence of LHRE_ApoA1_h_6):
5′-GATCTTGGGGCTGGGCAAATAGAGTGGGCAAG-3′
SEQ ID NO:15
(sense sequence of LHRE_ApoA1_h_7):
5′-GATCCGGGACAGAGCTGATCCTTGAACTA-3′
SEQ ID NO:16
(antisense sequence of LHRE_ApoA1_h_7):
5′-GATCTAGTTCAAGGATCAGCTCTGTCCCG-3′
SEQ ID NO:17
(sense sequence of LHRE_ApoA1_h_8):
5 -GATCCAGCTTGCTGTTTGCCCACTCTATA-3′
SEQ ID NO:18
(antisense sequence of LHRE_ApoA1_h_8):
5′-GATCTATAGAGTGGGCAAACAGCAAGCTG-3′
SEQ ID NO:19
(sense sequence used for mutagenesis of
ABCmutLuc+):
5′-ggacagagctgattgttgaactcttaagttccacattgcc-3′
SEQ ID NO:20
(antisense sequence used for mutagenesis of
ABCmutLuc+):
5′-cttaagagttcaacaatcagctctgtccctggggctgg-3′
SEQ ID NO:21
(sense sequence of FXRRE_ApoA1_h_1):
5′-CAGAGCTGATCCTTGAACTCTTAAGTT-3′
SEQ ID NO:22
(antisense sequence of FXRRE ApoA1_h_1):
5′-AACTTAAGAGTTCAAGGATCAGCTCTG-3′
SEQ ID NO:23
(sense sequence of FXRRE_ApoA1_h_1_mut):
5′-CAGAGCTGATCCTTGAAGTGTTAAGTT-3′
SEQ ID NO:24
(antisense sequence of FXRRE_ApoA1_h_1_mut):
5′-AACTTAACACTTCAAGGATCAGCTCTG-3′
SEQ ID NO:25
(sense sequence of LRHRE-ApoA1_mut):
5′-GATCCGGGACAGAGCTGATTGTTGAACTA-3′
SEQ ID NO:26
(antisense sequence of LRHRE-ApoA1_mut):
5′-GATCTAGTTCAACAATCAGCTCTGTCCCG-3′

### EXAMPLES

### Example 1

### Effect of LRH-1 Overexpression on the Activity of the Human apo AI Promoter in HepG2 Cells.

Example 1 shows that overexpression of hLRH-1 increased the activity of fragment −254/+91 (which contains regions A, B and C) of the apo AI promoter, cloned upstream of the luciferase reporter gene.

HepG2 cells were cotransfected by the lipofection technique (JetPEI according to the supplier's instructions) with 100 ng of the LRH-1 overexpression vector pCI-hLRH-1 or the empty vector pCI used as negative control and 250 ng of a reporter vector ABCLuc+ expressing the luciferase reporter gene under the control of fragment −254/+91 of the apo Al promoter (comprising regions A, B, C of the hApo Al promoter) (ABCLuc+) or 250 ng of promoter-free reporter vector as control (Luc+). These constructs were obtained by exchanging the CAT reporter gene from the previously described constructs [16] with the luciferase reporter gene extracted from Promega plasmid pGL3 (Madison, Wis., USA) as previously described [17]. The total amount of transfected DNA was adjusted to 500 ng using the pBKS+ plasmid. After 3 hours of transfection, cells were incubated in culture medium for 36 hours. Luciferase activity was then measured as previously described in the presence or absence of the LRH-1 protein.

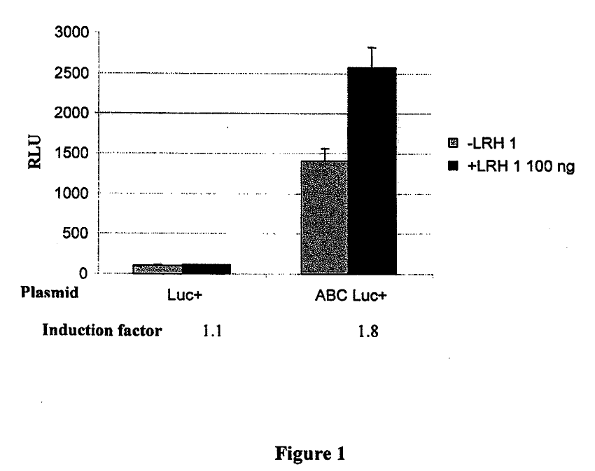

FIG. 1 shows that LRH-1 overexpression resulted in a two-fold increase in luciferase activity when HepG2 cells were transfected with the ABCLuc+ construct. This increase was not observed when cells were transfected with the promoter-free control construct Luc+.

### Example 2

### Effect of LRH-1 Overexpression on the Activity of the Human apo AI Promoter in RK13 Cells.

Example 2 shows that overexpression of hLRH-1 increased the activity of fragments −254/+91 (which contains regions A, B and C) and −192/+21 (which contains regions B and C) of the apo AI promoter but not of fragments −128/+91 (which contains region C) and −40/+91 (which contains only the minimal promoter), cloned upstream of the luciferase reporter gene.

RK13 cells were cotransfected by the lipofection technique (JetPEI according to the supplier's instructions) with 100 ng of the LRH-1 overexpression vector pCI-hLRH-1 or the empty vector pCI used as negative control and 250 ng of a reporter vector ABCLuc+ expressing the luciferase reporter gene under the control of fragments −254/+91 (comprising regions A, B, C of the hApo AI promoter; ABCLuc+), −192/+91 (comprising regions B and C; BCLuc+), −128/+91 (comprising region C; CLuc+) or −40/+91 (comprising the minimal promoter; pmin) of the apo AI promoter or 250 ng of promoter-free reporter vector as control (Luc+). These constructs were obtained by exchanging the CAT reporter gene from the previously described constructs [16] with the luciferase reporter gene extracted from Promega plasmid pGL3 (Madison, Wis., USA) as previously described [17]. The total amount of transfected DNA was adjusted to 500 ng using the pBKS+ plasmid. After 3 hours of transfection, cells were incubated in culture medium for 36 hours. Luciferase activity was then measured as previously described [17] in the presence or absence of the LRH-1 protein

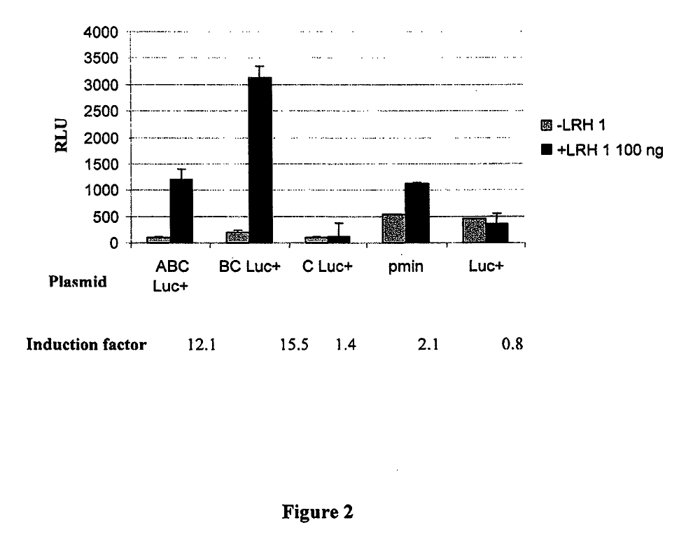

FIG. 2 shows that in the presence of LRH-1, expression of the luciferase gene placed under the control of the human apo AI promoter comprising regions A, B and C plus the minimal promoter (ABCLuc+; fragment −254/+91) was increased twelve-fold in RK13 cells which do not endogenously express the LRH-1 receptor. Luciferase expression under the control of a construct comprising regions B, C plus the minimal promoter (BCLuc+; fragment −192/+91) of the human apo AI promoter was increased by a factor of 15. Luciferase expression under the control of a construct comprising region C plus the minimal promoter (CLuc+; fragment −128/+91) of the human apo AI promoter was unaffected. Luciferase expression under the control of only the minimal promoter (pmin; fragment −40/+91) of the human apo AI promoter was only slightly increased by a factor of 2.

Luciferase expression was unchanged when the RK13 cells were transfected with the promoter-free vector Luc+.

The expression of the apo AI gene is therefore regulated by the LRH-1 protein. There is a cis-acting site located in region B of the human apo AI gene promoter allowing trans binding of the LRH-1 protein.

### Example 3

### Identification of an LRH-1 Binding Site in the Human apo AI Promoter.

Example 3 shows that LRH-1 binds to fragment −144/−122 of the human apo AI gene promoter.

The hLRH-1 protein was produced in vitro with the TNT-T7 rabbit reticulocyte lysate kit from Promega (ref. L4610) and the vector pCI-LRH-1. Double-stranded oligonucleotides corresponding to the LRH-1 response element present in the Cyp7a gene (hCyp7a wt), [sense: 5′-GATCTCTTAGTTCAAGGCCAGTTAG-3′ (SEQ ID NO: 7) and antisense: 5′-GATCCTAACTGGCCTTGAACTAAGA-3′ (SEQ ID NO:8)], to the same mutated reponse element hCyp7a mut, [sense: 5′-GATCTCTTAGTTCAATTCCAGTTAG-3′ (SEQ ID NO: 9) and antisense: 5′-GATCCTAACTGGAATTGAACTAAGA-3′ (SEQ ID NO: 10], to fragment −191/−171 (LRHPE_ApoA1_h_{—}5), [sense: 5′-GATCCGCAGCCCCCGCAGCTTGCTGTA-3′ (SEQ ID NO: 11) and antisense: 5′-GATCTACAGCAAGCTGCGGGGGCTGCG-3′ (SEQ ID NO: 12)], to fragment −178/−145 (LRHRE_ApoA1_h_{—}6), [sense: 5′-GATCCTTGCCCACTCTATTTGCCCAGCCCCAA-3′ (SEQ ID NO: 13) and antisense: 5′-GATCTTGGGGCTGGGCAAATAGAGTGGGCAAG-3′ (SEQ ID NO: 14)], to fragment −144/−122 wild type (LRHRE_ApoA1_h_{—}7), or mutated (LRHRE_ApoA1 mut), [respectively sense: 5′-GATCCGGGACAGAGCTGATCCTTGAACTA-3′ (SEQ ID NO: 15) and antisense: 5′-GATCTAGTTCAAGGATCAGCTCTGTCCCG-3′ (SEQ ID NO: 16), sense: 5′-GATCCGGGACAGAGCTGATTGTTGAACTA -3′ (SEQ ID NO: 25) and antisense: 5′-GATCTAGTTCAACAATCAGCTCTGTCCCG -3′ (SEQ ID NO: 26)] and to fragment −180/−158 (LRHRE_ApoA1_h_{—}8), [sense: 5′-GATCCAGCTTGCTGTTTGCCCACTCTATA-3′ (SEQ ID NO: 17) and antisense: 5′-GATCTATAGAGTGGGCAAACAGCAAGCTG-3′ (SEQ ID NO: 18)] of the human apo AI gene promoter (SEQ ID NO: 5) were prepared as previously described [16], and labelled with [γ-³²P]-ATP using polynucleotide kinase.

2 μl of rabbit reticulocyte lysate programmed by hLRH-1 were incubated for 15 minutes at room temperature in a final volume of 20 μl of buffer containing 10 mM HEPES, 2.5 mM MgCl₂, 10% glycerol, 2.5 mg/ml BSA, 50 mM NaCl and 0.5 mM DTT with 2.5 μg of polydl-dC and 1 μg of herring sperm DNA in the presence of the labelled double-stranded oligonucleotides (0.5 ng). The complexes were then separated by non-denaturing gel electrophoresis in TBE 0.25X buffer.

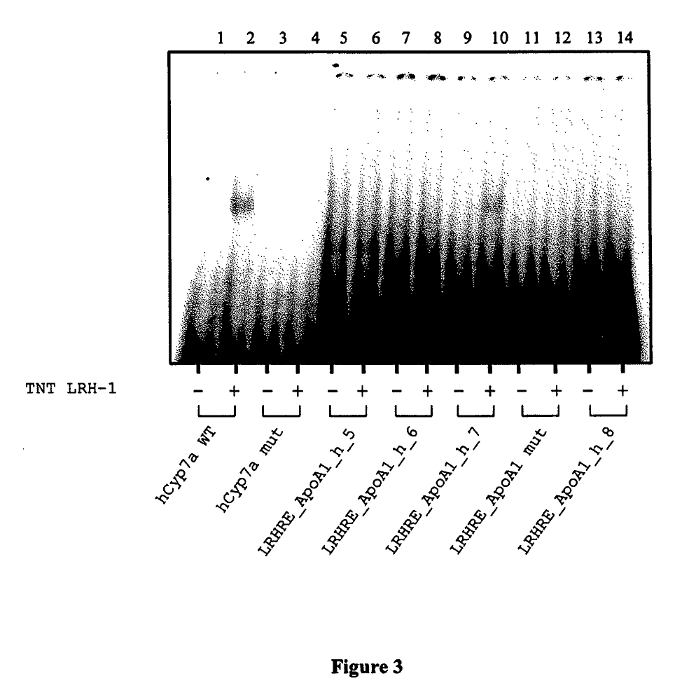

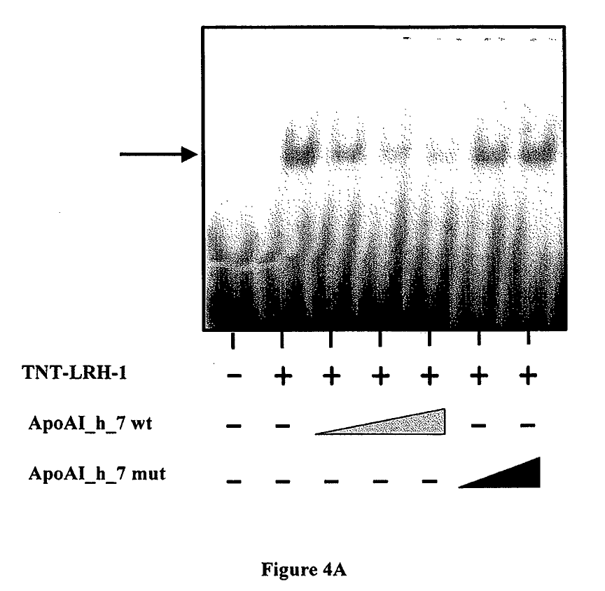

FIG. 3 shows that a specific LRH-1/DNA complex formed when the LRH-1 protein produced in vitro was incubated with a labelled double-stranded oligonucleotide corresponding to the LRH-1 response element in the Cyp7a gene (hCyp7a wt). In contrast, no complex was detected in the presence of labelled double-stranded oligonucleotide corresponding to the mutated response element (hCyp7a mut). FIG. 3 also shows that no DNA/LRH-1 complexes were seen with double-stranded oligonucleotides corresponding to fragments −191/−171 (LRHRE_ApoA1_h_{—}5), −178/−145 (LRHRE_ApoA1_h_{—}6), and −180/−158 (LRHRE_ApoA1_h_{—}8), of the human apo AI gene promoter. On the other hand, a specific LRH-1/DNA complex formed with a labelled double-stranded oligonucleotide corresponding to fragment −144/−122 (LRHRE_ApoA1_h_{—}7) of the human apo AI gene promoter. Said fragment straddles regions B and C of the human apo AI promoter and contains, on the antisense strand, the sequence TCAAGGATC similar to the consensus sequence TCAAGGTCA of an LRH-1 response element. Said element is functional since FIG. 3 shows that the corresponding double-stranded oligonucleotide in which the sequence TCAAGGATC was mutated (TCAACAATC) was unable to form a complex with LRH-1 (LRHRE_ApoA1 mut).

### Example 4

### Fragment −144/−122 of the Human apo AI Gene Promoter is a Low Affinity LRH-1 Response Element.

Example 4 shows that fragment −144/−122 of the human apo AI gene promoter is a low affinity binding site for LRH-1.

The hLRH-1 protein was produced in vitro with the TNT-T7 rabbit reticulocyte lysate kit from Promega (ref. L4610) and the vector pCI-LRH-1. Double-stranded oligonucleotides corresponding to the LRH-1 response element present in the Cyp7a gene (LRH-1-probe Cyp7a) or to the wild-type fragment −144/−122 (ApoA1_h_{—}7) of the human apo AI gene promoter (SEQ ID NO: 4) were prepared as previously described [16], and labelled with [γ-³²P]-ATP using polynucleotide kinase. 2 μl of reticulocyte lysate programmed by hLRH-1 were incubated for 15 minutes at 4° C. in a final volume of 20 μl of buffer containing 10 mM HEPES, 2.5 mM MgCl₂, 10% glycerol, 2.5 mg/ml BSA, 50 mM NaCl and 0.5 mM DTT with 2.5 μg of polydI-dC and 1 μg of herring sperm DNA in the presence of unlabelled double-stranded oligonucleotides in excess (10X, 50X and 100X) relative to the labelled probe (0.5 ng). Labelled double-stranded oligonucleotides (0.5 ng) were then added to the mixture and incubated at room temperature for 15 minutes. The complexes were then separated by non-denaturing gel electrophoresis in TBE 0.25X buffer.

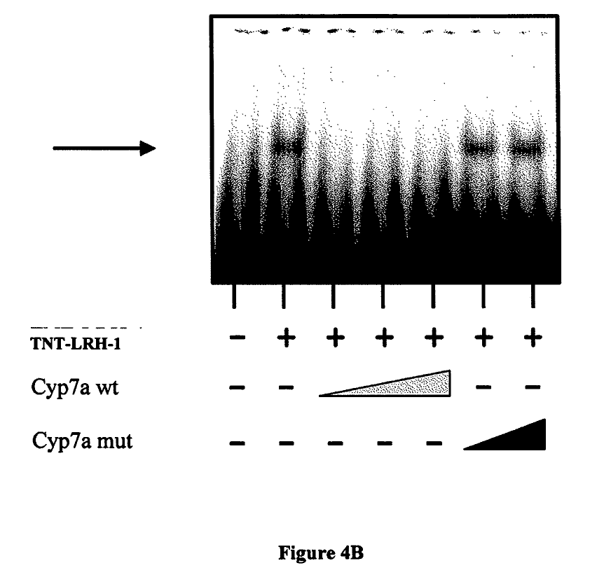

FIG. 4A shows that an unlabelled double-stranded oligonucleotide corresponding to fragment −144/−122 of the human apo AI gene promoter partially displaced the complex formed between LRH-1 and a labelled double-stranded oligonucleotide corresponding to the LRH-1 response element in the Cyp7a gene. On the other hand, FIG. 4A shows that there was no displacement of the complex formed between LRH-1 and a labelled double-stranded oligonucleotide corresponding to the LRH-1 response element in the Cyp7a gene by an unlabelled double-stranded oligonucleotide corresponding to mutated fragment −144/−122 of the human apo AI gene promoter.

FIG. 4B shows that an unlabelled double-stranded oligonucleotide corresponding to the LRH-1 response element in the Cyp7a gene totally displaced the complex formed between LRH-1 and a labelled double-stranded oligonucleotide corresponding to fragment −144/−122 of the human apo AI gene promoter. On the other hand, FIG. 4B shows that there was no displacement of the complex formed between LRH-1 and a labelled double-stranded oligonucleotide corresponding to fragment −144/−122 of the human apo AI gene promoter by an unlabelled double-stranded oligonucleotide corresponding to the mutated LRH-1 response element present in the Cyp7a gene. A comparison of FIGS. 4A and B indicates that the affinity of LRH-1 for fragment −144/−122 of the human apo AI gene promoter was lower than that for the LRH-1 response element present in the Cyp7a gene.

### Example 5

Effect of LRH-1 Overexpression on the Activity of the Human apo AI Promoter, Mutated or not, in HuH7 Cells.

Example 5 shows that mutation of the site TCAAGGATC present in fragment −144/−122 of the human apo AI gene promoter reduced the sensitivity to LRH-1 of a construct containing fragment −254/+91 of the human apo AI gene promoter.

HuH7 cells were cotransfected by the lipofection technique (JetPEI according to the supplier's instructions) with 100 ng of the LRH-1 overexpression vector pCI-hLRH-1 or the empty vector pCI used as negative control and 50 ng of a reporter vector ABCLuc+ expressing the luciferase reporter gene under the control of fragment −254/+91 comprising wild-type regions A, B, C of the Apo AI promoter (ABCLuc+), under the control of fragment −254/+91 comprising regions A, B and C of the apo AI promoter in which the site TCAAGGATC was mutated (ABCmutLuc+), under the control of fragment −192/+91 comprising regions B and C of the hApo AI promoter (BCLuc+), under the control of fragment −128/+91 comprising region C of the apo AI promoter, (CLuc+) or under the control of fragment −40/+91 comprising the minimal apo AI promoter (pmin) or 50 ng of promoter-free reporter vector as control (Luc+). The mutant construct ABCmutLuc+ was obtained by site-directed mutagenesis of the wild-type ABCLuc+ construct, using the Quick Change Site Directed Mutagenesis kit (Stratagene) corresponding to the sense sequence SEQ ID NO 19 and the antisense sequence SEQ ID NO 20.

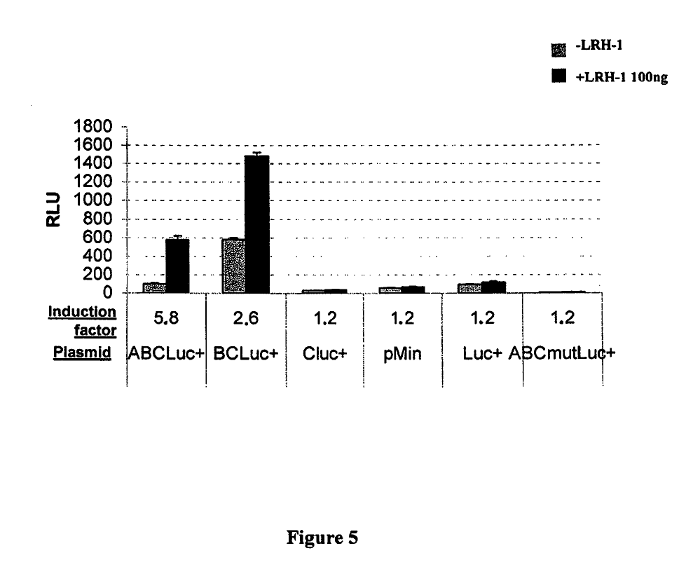

FIG. 5 shows that LRH-1 overexpression resulted in a 5.8-fold increase in luciferase activity when the HuH7 cells were transfered with the ABCLuc+ construct and a 2.6-fold increase when the BCLuc+ construct was used for transfection. There was little or no increase when cells were transfected with constructs comprising regions A, B and C of the apo AI promoter in which the site TCAAGGATC was mutated (ABCmutLuc+), or with constructs comprising region C of the apo AI promoter (Cluc+), the minimal apo AI promoter (pmin) or the promoter-free construct (Luc+).

Example 5 shows that the TCAAGGATC site present in fragment −144/−122 of the human apo AI gene promoter sensitizes it to LRH-1.

### Example 6

### Effect of LRH-1 Overexpression on the Activity of Different Human apo AI Promoter Mutants in HuH7 Cells.

Example 6 shows that mutation of the TCAAGGATC site present in fragment −144/−122 reduced the sensitivity to LRH-1 of a contruct comprising fragment −254/+91 of the human apo AI gene promoter cloned upstream of the luciferase reporter gene, in contrast to mutation of the adjacent FXR response element of the human apo AI promoter.

HuH7 cells were cotransfected by the lipofection technique (JetPEI according to the supplier's instructions) with 100 ng of the LRH-1 overexpression vector pCI-hLRH-1 or the empty vector pCI used as negative control and 50 ng of a reporter vector expressing the luciferase reporter gene under the control of fragment −254/+91 comprising wild-type regions A, B nd C of the apo AI promoter (ABCLuc+and ABCpGL3), under the control of fragment −254/+91 comprising regions A, B and C of the apo AI promoter in which the site TCAAGGATC was mutated (ABCmutLuc+—see example 5), under the control of fragment −254/+91 comprising regions A, B and C of the apo AI promoter in which the FXR response element was mutated (ABCpGL3FXREKO), under the control of fragment −192/+91 comprising regions B and C of the hApo AI promoter (BCLuc+ and BCpGL3), under the control of fragment −192/+91 in which the FXR response element was mutated (BCpGL3FXREKO), under the control of fragment −128/+91 comprising region C of the apo AI promoter (Cluc+), under the control of fragment −40/+91 comprising the minimal apo AI promoter (pmin) or 50 ng of promoter-free reporter vector as control (Luc+ and pGL3).

The ABCpGL3 construct was obtained by subcloning fragment −254/+91 of the apo AI promoter from the ABCLuc+ vector (Sa1I/SphI digestion) into the pGL3 vector from Promega (XhoI/SphI digestion). The ABCpGL3FXREKO construct was obtained by subcloning the mutated fragment −254/+91 of the apo AI promoter from the ABCLuc+FXREKO vector described previously [18] (SaII/SphI digestion) into the pGL3 vector (XhoI/SphI digestion). The BCpGL3 and BCpGL3FXREKO constructs were obtained by partial digestion and religation of ABCpGL3 and ABCpGL3FXREKO, respectively.

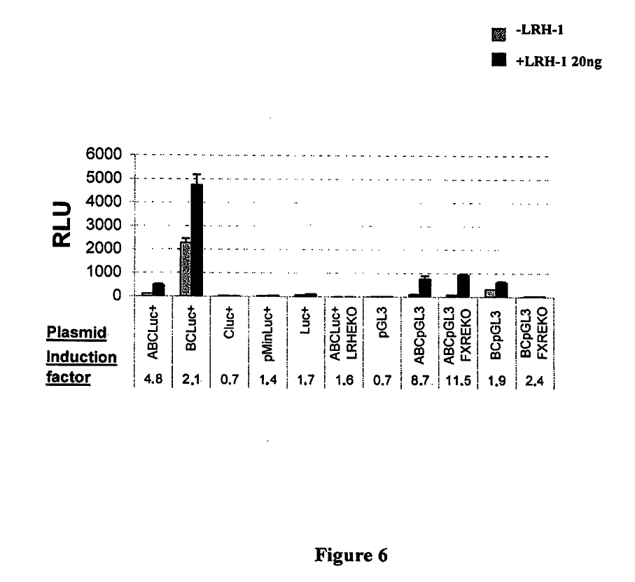

FIG. 6 shows that LRH-1 overexpression increased luciferase activity by a factor of 4.8 when HuH7 cells were transfected with the ABCLuc+ construct, by a factor of 2.1 after transfection with BCLuc+, 8.7 after transfection with ABCpGL3, 11.5 for ABCpGL3FXREKO, 1.9 for BCpGL3 and 2.4 after transfection with BCpGL3FXREKO. Little or no increase was observed when the cells were transfected with constructs comprising regions A, B and C of the apo AI promoter in which the TCAAGGATC was mutated (ABCmutLuc+), or with constructs comprising region C of the apo AI promoter (Cluc+), the minimal apo AI promoter (pmin) or the promoter-free constructs (Luc+and pGL3).

Example 6 shows that the TCAAGGATC site present in fragment −144/−122 of the human apo AI gene promoter sensitizes it to LRH-1. The presence of the FXR response element adjacent to the LRH response element did not appear necessary for a response to LRH.

Therefore, these two response elements, though physically close, are functionally distinct.

### Example 7

The LRH-1 Binding Site in the Human apo I Gene Promoter is Different from the FXR Binding Site in the Human apo AI Gene Promoter.

Example 3 shows that LRH-1 binds to fragment −144/−122 of the human apo AI gene promoter.

Example 7 shows that the binding site of LRH-1 on the human apo AI gene promoter is different from the binding site of FXR on this same promoter.

LRH-1 and FXR proteins were produced in vitro with TNT-T7 (rabbit reticulocyte lysate, Promega, ref. L4610) and the vectors pCI-LRH-1 and pCDNA3-FXR.

Gel shift experiments were carried out as described in example 3 with double-stranded oligonucleotides corresponding to:

the LRH-1 response element present in the Cyp7a gene (hCyp7a wt), [sense: 5′-GATCTCTTAGTTCAAGGCCAGTTAG-3′ (SEQ ID NO: 7) and antisense: 5′-GATCCTAACTGGCCTTGAACTAAGA-3′ (SEQ ID NO:8)], the FXR response element of the human apo AI gene promoter (FXRRE_ApoA1_h_{—}1), [sense: 5′-CAGAGCTGATCCTTGAACTCTTAAGTT-3′ (SEQ ID NO: 21) and antisense: 5′-AACTTAAGAGTTCAAGGATCAGCTCTG-3′ (SEQ ID NO: 22)], the same response element in mutated form (FXRRE_ApoA1_h_{—}1_mut), [sense: 5′-AACTTAAGAGTTCAAGGATCAGCTCTG-3′ (SEQ ID NO: 23) and antisense: 5′-AACTTAACACTTCAAGGATCAGCTCTG-3′ (SEQ ID NO: 24], and fragment −144/−122 of the wild-type human apo AI gene promoter (LRHRE_ApoA1_h_{—}7), or mutated (LRHRE_ApoA1 mut), [respectively sense: 5′-GATCCGGGACAGAGCTGATCCTTGAACTA-3′ (SEQ ID NO: 15) and antisenss: 5′-GATCTAGTTCAAGGATCAGCTCTGTCCCG-3; ′ (SEQ ID NO: 16), sense: 5′-GATCCGGGACAGAGCTGATTGTTGAACTA -3′ (SEQ ID NO: 25) and antisense: 5′-GATCTAGTTCAACAATCAGCTCTGTCCCG-3′ (SEQ ID NO: 26)].

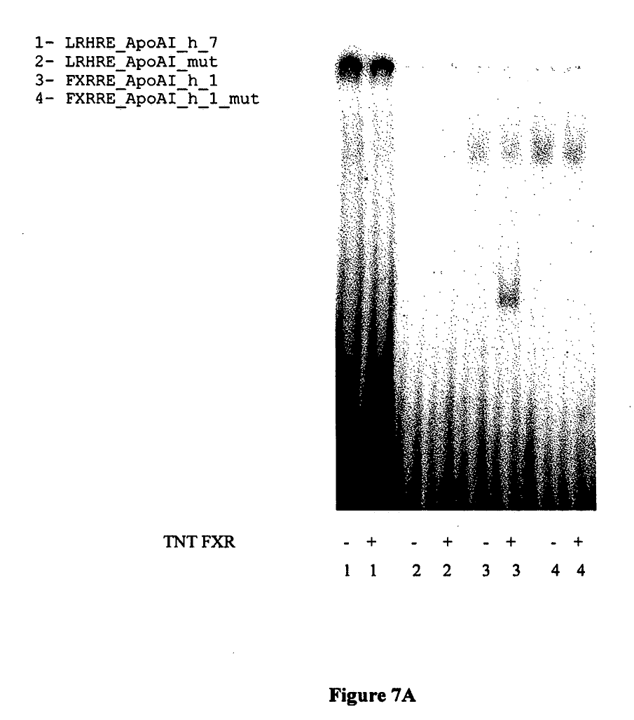

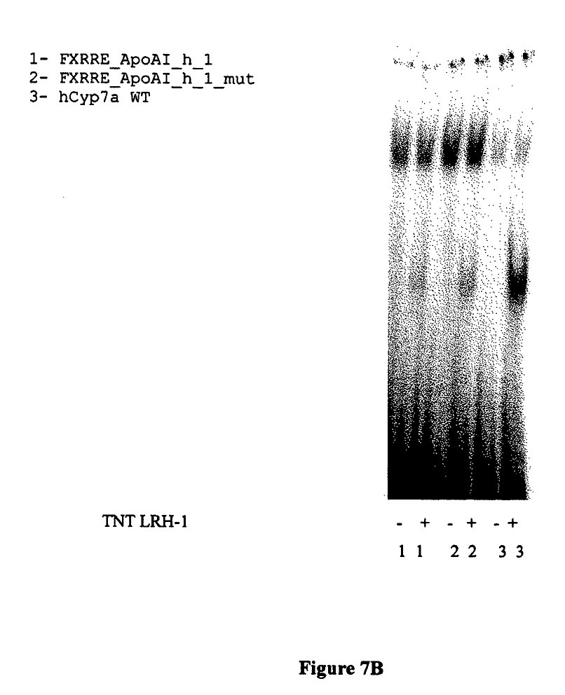

FIGS. 3 and 7B show that a specific LRH-1/DNA complex formed when the LRH-1 protein produced in vitro was incubated with a labelled double-stranded oligonucleotide corresponding to the LRH-1 response element in the Cyp7a gene (hCyp7a wt). On the other hand, no complexes were detected with the labelled double-stranded oligonucleotide corresponding to the mutated response element (hCyp7a mut) (FIG. 3). FIG. 3 also shows that a specific LRH-1/DNA complex formed when the LRH-1 protein was incubated with the labelled double-stranded oligonucleotide corresponding to fragment −144/−122 (LRHRE_ApoA1_h_{—}7) of the human apo AI gene promoter whereas said complex was absent with the corresponding double-stranded oligonucleotide in which the sequence TCAAGGATC was mutated (LRHRE_ApoA1 mut).

FIG. 7A shows that no FXR/DNA complexes were detected when the FXR protein produced in vitro was incubated with labelled double-stranded oligonucleotides corresponding to fragment −144/−122 (LRHRE_ApoA1_h_{—}7) of the human apo AI gene promoter or with the same mutated fragment (LRHRE_ApoA1 mut). On the other hand, FIG. 7A shows the presence of a specific FXR/DNA complex when the FXR protein produced in vitro was incubated with a labelled double-stranded oligonucleotide corresponding to the FXR response element of the human apo AI gene promoter FXRRE_ApoA1_h_{—}1) whereas this complex was no longer present when the FXR protein was incubated with the same mutated response element (FXRRE_ApoA1_h_{—}1_mut).

FIG. 7B shows that a specific LRH-1/DNA complex formed when the LRH-1 protein produced in vitro was incubated with a labelled double-stranded oligonucleotide corresponding to the FXR response element of the human apo AI gene promoter (FXRRE_ApoA1_h_{—}1) or with the same mutated response element (FXRRE_ApoA1_h_{—}1_mut).

Thus, mutation of the FXR response element in the human apo AI gene promoter did not affect the binding of LRH-1 to its response element whereas said mutation affected the binding of FXR to its response element in the human apo AI gene promoter. Example 7 therefore demonstrates that the binding sites for LRH-1 and FXR in the human apo AI gene promoter are distinct.

### REFERENCES

1. Francis, G. A., et al., Nuclear receptors and the control of metabolism. Annu Rev Physiol, 2003. 65: p. 261-311.
2. Zhang, C. K., et al., Characterization of the genomic structure and tissue-specific promoter of the human nuclear receptor NR5A2 (hB1F) gene. Gene, 2001. 273(2): p. 239-49.
3. Sirianni, R., et al., Liver receptor homologue-1 is expressed in human steroidogenic tissues and activates transcription of genes encoding steroidogenic enzymes. J Endocrinol, 2002. 174(3): p. R13-7.
4. Ellinger-Ziegelbauer, H., et al., FTZ-F1-related orphan receptors in Xenopus laevis: transcriptional regulators differentially expressed during early embryogenesis. Mol Cell Biol, 1994. 14(4): p. 2786-97.
5. Lin, W., et al., Zebrafish ftz-fI gene has two promoters, is alternatively spliced, and is expressed in digestive organs. Biochem J, 2000. 348 Pt 2: p. 439-46.
6. Chen, F., et al., Liver Receptor Homologue-1 Mediates Species- and Cell Line-specific Bile Acid-dependent Negative Feedback Regulation of the Apical Sodium-dependent Bile Acid Transporter. J Biol Chem, 2003. 278(22): p. 19909-16.
7. Schoonjans, K., et al., Liver receptor homolog 1 controls the expression of the scavenger receptor class B type L EMBO Rep, 2002. 3(12): p. 1181-7.
8. Le Goff, W., et al., A CYP7A promoter binding factor site and Alu repeat in the distal promoter region are implicated in regulation of human CETP gene expression. J Lipid Res, 2003. 44(5): p. 902-10.
9. Segrest, J., et al., Structure and function of apolipoprotein A-I and high-density lipoprotein. Curr Opin Lipidol, 2000. 11(2): p. 105-15.
10. Fruchart, J. and P. Duriez, High density lipoproteins and coronary heart disease. Future prospects in gene therapy. Biochimie, 1998. 80(2): p. 167-72.
11. Leroy, A., J. Dallongeville, and J. Fruchart, Apolipoprotein A-I-containing lipoproteins and atherosclerosis. Curr Opin Lipidol, 1995. 6(5): p. 281-5.
12. Breslow, J., et al., Isolation and characterization of cDNA clones for human apolipoprotein A-I. PNAS, 1982. 79(22): p. 6861-6865.
13. Shoulders, C. and F. Baralle, Isolation of the human HDL apolipoprotein AI gene. Nucleic Acids Res, 1982. 10(16): p. 4873-4882.
14. Karathanasis, S., et al., Linkage of human apolipoproteins A-I and C-III genes.

Nature, 1983. 304(5924): p. 371-3.
15. Widom, R., et al., Synergistic interactions between transcription factors control expression of the apolipoprotein AI gene in liver cells. Mol Cell Biol, 1991. 11(2): p. 677-687.
16. Vu-Dac, N., et al., Negative regulation of the human apolipoprotein A-I promoter by fibrates can be attenuated by the interaction of the peroxisome proliferator-activated receptor with its response element. J Biol Chem, 1994. 269(49): p. 31012-31018.
17. Raspe, E., et al., Modulation of rat liver apolipoprotein gene expression and serum lipid levels by tetradecylthioacetic acid (TTA) via PPAR{alpha} activation. J. Lipid Res, 1999. 40(11): p. 2099-2110.
18. Claudel, T., et al., Bile acid-activated nuclear receptor FXR suppresses apolipoprotein A-I transcription via a negative FXR response element. J Clin Invest, 2002. 109(7): p. 961-971.

1-32. (canceled)

33. A method for the selection, identification or characterization of compounds which can modulate reverse cholesterol transport, which comprises:
a. contacting a test compound with a nucleic acid construct comprising, as the only LRH-1 response element, at least one copy of the LRH-1 response element of the promoter of the human apolipoprotein Al gene containing the following sequence (SEQ ID NO: 1): 5′-CTGATCCTTGAAC-3′, and
b. determining the possible binding of said test compound to the response element.

34. The method according to claim 33, wherein the contact is carried out in the presence of the exogenous LRH-1 receptor or a functional equivalent thereof, and in that one determines the possible binding of said test compound to the LRH-1 response element and/or to the complex formed by the binding of LRH-1 to its response element.

35. A method for the selection, identification or characterization of compounds which can modulate reverse cholesterol transport, which comprises:
a. contacting a test compound with a host cell containing a reporter gene expression cassette, said cassette comprising a reporter gene placed under the control of a promoter comprising, as the only LRH-1 response element, at least one copy of the LRH-1 response element of the promoter of the human apolipoprotein AI gene containing the following sequence (SEQ ID NO: 1): 5′-CTGATCCTTGAAC-3′, and
b. determining the effect of the presence of the test compound on the binding of LRH-1 to the resposne element or on the expression of the reporter gene.

36. The method according to claim 35, wherein the host cell comprises an exogenous LRH-1 receptor or a functional equivalent thereof.

37. The method according to claim 35, wherein the host cell comprises a ligand of LRH-1.

38. The method according to claim 35 comprising determining the level of expression of the reporter gene in the presence of the test compound and in the absence of said compound, an increase or a decrease in the level of reporter gene expression indicating the ability of the test compound to modulate reverse cholesterol transport.

39. The method according to claim 35, wherein the host cell is a mammalian cell.

40. The method according to claim 35, wherein the host cell is a human cell.

41. The method according to claim 35, wherein the reporter gene is a gene coding for a product whose activity or presence in biological extracts can be measured, in particular one of the genes coding for luciferase, secreted alkaline phosphatase, galactosidase or lactamase.

42. The method according to claim 35, wherein the promoter is selected in the group consisting of the HSV-TK promoter, the CMV immediate early promoter, the PGK promoter, the promoter of the gene coding for human apolipoprotein AI and the SV40 promoter.

43. The method according to claim 33, wherein one or more compounds are tested, as a mixture or separately.

44. The method according to claim 33, wherein the test compound is a combinatorial library.

45. The method according to claim 33, wherein the test compound is a clone or a library of nucleic acid clones coding for one or more DNA-binding polypeptide(s).

46. The method according to claim 33, wherein contact is carried out in a multiwell plate.

47. The method according to claim 33, wherein additionally comprising a comparison of the possible effects determined by said method with the possible effects determined by a method carried out in the same conditions but with a nucleic acid construct containing at least one mutated copy of the LRH-1 response element of the promoter of the human apolipoprotein AI gene, containing the following sequence (SEQ ID NO: 1): 5′-CTGATCCTTGAAC-3′, said mutant copy essentially being unable to bind the LRH-1 receptor.

48. The method according to claim 33, for the selection, identification or characterization of compounds which can increase reverse cholesterol transport.

49. The method according to claim 33, for the selection, identification or characterization of compounds which can modulate the activity of HDL.

50. The method according to claim 33, for the selection, identification or characterization of compounds which can modulate the expression of apolipoprotein AI.

51. A method for the modulation of reverse cholesterol transport, comprising administering to a subject in need thereof a compound which can modulate the binding of LRH-1 and/or its cofactors to the response element of the promoter of the human apolipoprotein gene or a functional variant thereof.

52. A method for the increase of reverse cholesterol transport, comprising administering to a subject in need thereof a compound increasing the binding of LRH-1 and/or its cofactors to the sequence SEQ ID NO: 1 or a functional variant thereof.

53. A method for the modulation of the activity of HDL, comprising administering to a subject in need thereof a compound modulating the binding of LRH-1 and/or its cofactors to the sequence SEQ ID NO: 1 or a functional variant thereof.

54. A method for the increase of the activity of HDL, comprising administering to a subject in need thereof a compound increasing the binding of LRH-1 and/or its cofactors to the sequence SEQ ID NO: 1 or a functional variant thereof.

55. A method for modulating the expression of ApoAI, comprising administering to a subject in need thereof a compound modulating the binding of LRH1 and/or its cofactors to the sequence SEQ ID NO: 1 or a functional variant thereof.

56. A method for the modulation of reverse cholesterol transport, comprising administering to a subject in need thereof a compound increasing the effect of LRH-1 and/or its cofactors on the transcription of the human apolipoprotein AI gene.

57. The method according to claim 51, characterized in that the compound is a nuclear factor or a cofactor.

58. The method according to claim 51, characterized in that the compound is a clone expressing one or more DNA-binding polypeptide(s).

59. The method according to claim 51, characterized in that the compound is a compound which is selected, identified or characterized by:
a. contacting a test compound with a nucleic acid construct comprising, as the only LRH-1 response element, at least one copy of the LRH-1 response element of the promoter of the human apolipoprotein AI gene containing the following sequence (SEQ ID NO: 1): 5′-CTGATCCTTGAAC-3′, and
b. determining the possible binding of said test compound to the response element.

60. A nucleic acid fragment characterized by the following sequence (SEQ ID NO: 1): 5′-CTGATCCTTGAAC-3′.

61. An expression cassette comprising at least one copy of the nucleic acid fragment as defined in claim 60, and a promoter, selected from among the CMV immediate early promoter and the PGK promoter, associated with a reporter gene placed under the control of said promoter.

62. A method for in vitro screening of compounds which can modulate the activity of HDL, wherein the cassette as defined in claim 61 is used.

63. A pharmaceutical composition, comprising a compound which is selected, identified or characterized according to claim 33.
